(19) United States
(12) Patent Application Publication
(10) Pub. No.: US 2012/0295306 A1
(43) Pub. Date: Nov. 22, 2012

(54) Modified CIPA Gene From Clostridium Thermocellum for Enhanced Genetic Stability

(75) Inventors: Devin Hedley Currie, Litchfield, NH (US); John McBride, Lyme, NH (US); Adam Guss, Etna, NH (US)

(73) Assignee: THE TRUSTEES OF DARTMOUTH COLLEGE, Hanover, NH (US)

(21) Appl. No.: 13/265,107

(22) PCT Filed: Apr. 21, 2010
(86) PCT No.: PCT/US10/31907

§ 371 Date: May 24, 2012

Related U.S. Application Data
(60) Provisional application No. 61/171,197, filed on Apr. 21, 2009.

(51) Int. Cl. C12N 15/74; C07H 21/04; C12N 15/63; C12P 21/00; C12N 1/21

(52) U.S. Cl. 435/69.1; 435/471; 435/252.3; 435/320.1; 536/23.1

(57) ABSTRACT

Bacteria consume a variety of biomass-derived substrates and produce ethanol. The scaffoldin gene cipA from Clostridium thermocellum is modified to generate a mutated gene with enhanced genetic stability. This mutated cipA gene can be introduced into a heterologous host, such as Thermoanaerobacterium saccharolyticum. Other cellulosome components may be introduced into the host to build a full-sized cellulosome in T. saccharolyticum. Manipulation of the scaffoldin genes provides a new approach for enhancing ethanol production by biomass-fermenting microorganisms.

### RELATED APPLICATIONS

This application claims priority of U.S. Provisional Application No. 61/171,197 filed on Apr. 21, 2009, the contents of which are hereby incorporated into this application by reference.

### SEQUENCE LISTING

This application is accompanied by a sequence listing in a computer readable form that accurately reproduces the sequences described herein.

### BACKGROUND

1. Field of the Invention

The present disclosure pertains to the field of biomass processing to produce ethanol. More particularly, the disclosure relates to modification of cellulosome components with enhanced stability and functionality.

2. Description of the Related Art

Lignocellulosic biomass represents one of the most abundant renewable resources on Earth. Lignocellulosic biomass generally contains three major components—cellulose, hemicellulose, and lignin. Some of the most common source of lignocellulosic biomass includes agricultural and forestry residues, municipal solid waste (MSW), fiber resulting from grain operations, waste cellulosics (e.g., paper and pulp operations), and energy crops. The cellulose and hemicellulose polymers of biomass may be hydrolyzed into their component sugars, such as glucose and xylose, which can then be fermented by microorganisms to produce ethanol. Conversion of even a small portion of the available biomass into ethanol could substantially reduce our dependence on fossil energy.

Cellulosomes are multienzyme systems used by some bacteria to degrade cellulose and hemicellulose. Cellulosomes are capable of degrading the polysaccharide components, most notably, cellulose and hemicellulose, in the cell walls of plants. The cellulosomes of the anaerobic thermophilic bacterium, Clostridium thermocellum, may contain as many as 100 or more enzymatic and non-enzymatic components. For review, see Lynd et al., (2002) “Microbial cellulose utilization: fundamentals and biotechnology.” Microbiol. Mol. Biol. Rev. 66:506-77; see also, Zverlov et al., (2005) “Functional subgenomics of Clostridium thermocellum cellulosomal genes: Identification of the major catalytic components in the extracellular complex and detection of three new enzymes.” Proteomics 5:3646-53.

One of the non-catalytic subunits in cellulosomes is called scaffoldin, which may help secure various enzymatic subunits into the complex via the cohesin-dockerin interaction. Cohesins are modules located on the scaffoldin subunits, while dockerins are domains on various enzymatic subunits. The specific interaction between the cohesin modules and the dockerin domains likely dictates the supramolecular architecture of the cellulosomes.

The C. thermocellum CipA protein contains nine type I cohesin modules to which enzymes and other protein components specifically dock by virtue of type I dockerin modules. See Zverlov et al., (2008) “Mutations in the Scaffoldin Gene, cipA, of Clostridium thermocellum with Impaired Cellulosome Formation and Cellulose Hydrolysis Insertions of a New Transposable Element, IS1447, and Implications for Cellulase Synergism on Crystalline Cellulose.” J. Bacteriol. vol. 192, p. 4321-27. In addition, cell wall-bound proteins, such as OlpB or SdbA help anchoring the CipA protein to the cell wall via type II cohesin-dockerin interactions. Leibovitz et al., (1997) “Characterization and subcellular localization of the Clostridium thermocellum scaffoldin dockerin binding protein SdbA.” J. Bacteriol. 179:2519-23; see also, Bayer et al. (1998) “Cellulosomes—structure and ultrastructure.” J. Struct. Biol. 124:221-34.

Although the cellulosomes of cellulolytic C. thermocellum are one of the best understood systems among bacteria, the regulation of the expression of the various components of the cellulosomes is not well understood. Another thermophilic bacterium, T. saccharolyticum, which does not have a cellulosome system, is capable of growing on a wider range of sugars, as compared to C. thermocellum. Because T. saccharolyticum is non-cellulolytic, it tends to generate less side products as a result of cellulose degradation than those generated by cellulolytic C. thermocellum. More importantly, T. saccharolyticum possesses almost all of the systems necessary to utilize hydrolysis products of cellulose, and is thus more efficient in utilizing biomass to produce ethanol.

If the cellulosome systems can be built in T. saccharolyticum, both the efficiency of the cellulosome system and the advantage of the T. saccharolyticum host can be employed to create an improved and efficient ethanol-producing organism. The cipA gene of C. thermocellum encodes a scaffoldin protein, which acts as the backbone for building cellulosomes. One major obstacle is posed by the presence of extensive repeated sequences in the cipA gene which may render the cipA gene unstable and difficult to clone. The repeated regions may cause errors in DNA replication and polymerase chain reaction (PCR). Moreover, the repeated regions may also cause truncation of the cipA gene due to homologous recombination in various hosts, such as yeast, E. coli, or T. saccharolyticum, among others.

### SUMMARY

The present instrumentalities advance the art by providing an important first step towards building an efficient fermentative microorganism for converting biomass into ethanol. More specifically, the present instrumentalities advance the art by providing a modified version of the cipA gene (“mcipA” hereinafter) which may act as the backbone for building a full-sized cellulosome system. The modified cipA gene contains much less repeated sequences than the wildtype cipA gene from C. thermocellum and is more stable when introduced into a cellulose-degrading organism such as T. saccharolyticum.

In one embodiment, the cellulosome systems of the present disclosure may be built in a host organism that possesses its native cellulosome system. In another embodiment, the cellulosome systems may be reconstructed in a host organism that does not have a native cellulosome system. Examples of such organisms include, but are not limited to, T. saccharolyticum. In one aspect, the de novo reconstruction of such a cellulosome system may be accomplished by stepwise introduction of various known components of the system into the host organism. One advantage of this stepwise approach is that the functionality and interaction between various components can be dissected in detail. Such engineered organisms may utilize a variety of biomass derived substrates to generate ethanol in high yields. In another aspect, multiple components, i.e., genes or proteins, may be introduced into the host organism at the same time to build the cellulosomes of the present disclosure.

In another embodiment, genes encoding anchor proteins, such as SdbA or CelS proteins from C. thermocellum, may be introduced into the host organism to act as anchor proteins on the cell wall of the host organism. When the coding sequences of such genes are expressed heterologously in another organism, cautions need to be taken to ensure accurate translation, folding, and secretion of the proteins onto the cell wall. Traceable tags, such as a 6×His tag, may be engineered into the expression vector such that the localization of the protein can be readily determined.

In one aspect, the CipA scaffoldin protein may be expressed in T. saccharolyticum to serve as the backbone for building the cellulosomes. To this end, the coding sequence of the cipA gene from C. thermocellum (SEQ ID. NO. 1) may be subcloned into an expression vector suitable for transcription and translation in T. saccharolyticum.

The coding sequence of the cipA gene of C. thermocellum contains extensive areas of repeated sequences which may render the gene unstable. For example, two large 470 base pair (bp) repeats exist, and numerous smaller repeats exist in the cipA gene. The ten biggest repeats by length on the cipA gene (also referred to as Repeat Groups 1-10) are shown in Table 1, with the relative position of each repeat sequence along the 5562 by full-length cipA gene indicated. Note that different Repeat Groups have different number of repeat sequences on the cipA gene. For instance, while Repeat Group #1 only has two repeat sequences on the cipA gene, Repeat Group #3 has four repeat sequences spread out on the cipA gene.

TABLE 1
The 10 largest Repeats on the coding sequence of cipA

| | Total number of repeats | Position on the 5562 bp | |
|---|---|---|---|
| Repeat | on the C. thermocellum | C. thermocellum | Length |
| # | cipA 5562 bp sequence | wildtype cipA sequence | (bp) |
| 1 | 2 | 2947-3416 | 470 |
| | | 3937-4406 | |
| 2 | 2 | 2452-2906 | 455 |
| | | 3937-4391 | |
| 3 | 4 | 2488-2825 | 338 |
| | | 2983-3320 | |
| | | 3478-3815 | |
| | | 3973-4310 | |
| 4 | 3 | 2185-2411 | 227 |
| | | 3175-3401 | |
| | | 4165-4391 | |
| 5 | 2 | 3769-3969 | 201 |
| | | 4756-4956 | |
| 6 | 2 | 3418-3598 | 181 |
| | | 4408-4588 | |
| 7 | 3 | 1924-2074 | 151 |
| | | 2413-2563 | |
| | | 2908-3058 | |
| 8 | 2 | 1846-1995 | 150 |
| 9 | 2 | 2185-2330 | 146 |
| 10 | 4 | 2488-2608 | 121 |
| | | 2983-3103 | |
| | | 3973-4093 | |
| | | 4468-4588 | |

To increase genetic stability and to avoid unwanted homologous recombination among these repeats, one or more of these repeated sequences may be removed. The repeats may be removed using various molecular biology tools. These techniques include but are not limited to restriction digestion, PCR, whole-gene synthesis or other methods for introducing silent mutations into the coding sequence of a gene. Silent mutations are mutations in a coding sequence that do not result in any changes in the sequence of the encoded protein. Caution is to be taken to ensure that no Stop (or nonsense) codon is introduced in the middle of the coding sequence during this process. More preferably, all major repeat sequences of the wildtype cipA gene (wcipA), namely, Repeats 1-10, as shown in Table 1, are eliminated by mutation. By way of example, one such modified cipA gene (mcipA), SEQ ID NO: 2, is disclosed.

In one aspect, a polynucleotide may be created and isolated which shares at least 70%, 80%, 90%, 95%, 98%, or 99% sequence identity with the polynucleotide of SEQ ID NO: 1, wherein at least one repeat sequence selected from Repeats #1-10 (as listed in Table 1) has been eliminated by mutation. For purpose of this disclosure, if either one or all of the repeat sequences belonging to any one of the Repeat Groups #1-10 are mutated such that none of these repeat sequences that originally belong to one Repeat Group share more than 99% sequence identity with one another, it can be said that this particular Repeat Group has been eliminated. For instance, if either one or both of the Repeat #1 sequences have been mutated so that they share 99% or less sequence identity with one another, it can be said that Repeat #1 has been eliminated. More preferably, all 10 Repeat Groups, Repeats 1-10, are removed so that all repeat fragments (or sequences) in each Repeat group share less than 99%, preferably less than 80%, 70%, 60% or, more preferably 50%, sequence identity with one another. Most preferably, all repeats on the cipA gene are removed without causing significant changes in the encoded protein such that the protein encoded by the modified gene shares at least 90%, 98%, 99%, or more preferably 100% amino acid sequence identity with the wildtype cipA protein (wCipAp) of SEQ ID NO: 3. In another aspect, other variants of mcipA with at least 80%, 90%, 95%, 98%, 99%, or more preferably 100% identity with the polynucleotide of SEQ ID NO: 2 may be used.

In another aspect, the cipA gene of C. thermocellum is modified by mutating the coding sequence such that the mutated gene utilizes alternative codons that are more commonly used in T. saccharolyticum. The present disclosure may thus provide a modified cipA gene which is optimized for T. saccharolyticum and which contain none or only minimum amount of repeated sequences.

In another embodiment of the present disclosure, various enzymes may be introduced into the host organism, preferably after the anchor and scaffoldin proteins are introduced and expressed. These enzymes include, but are not limited to, those cellulosome components encoded by the 72 genes disclosed in Zverlov (2005). These enzymes typically bear one or more dockerin modules which indicate of their association with the cellulosome systems.

In another embodiment, a genetic construct comprising a polynucleotide sequence having at least 70%, 80%, 90%, 95%, 98%, or 99% sequence identity with the sequence of SEQ ID NO: 1, and with at least one repeat sequence selected from Repeats 1-10 removed, said polynucleotide sequence being operably linked to a promoter capable of controlling transcription in a bacterial cell, is described. The promoter can be a constitutive or an inducible promoter. The polynucleotides which contain relatively less repeat sequences may be introduced into a cell or an organism where scaffolding proteins encoded by the modified polynucleotides may be expressed. More preferably, the promoter may enhance gene expression from said polynucleotide in the host cell or organism, such as Thermoanaerobacterium saccharolyticum.

In another embodiment, a genetically engineered cell expressing a scaffoldin encoded by a gene having at least 70%, 80%, 90%, 95%, 98%, or 99% identity with the sequence of SEQ ID NO: 1, and with at least one repeat sequence selected from Repeats 1-10 removed, the expression of said scaffoldin being driven by a heterologous promoter, is described. The promoter can be a constitutive or an inducible promoter.

In another embodiment, an organism capable of growing on a carbohydrate-rich biomass substrate may be generated. said organism comprising the polynucleotide of claim 1, wherein said organism is capable of expressing a scaffolding protein encoded by said polynucleotide.

In another embodiment, a method for improving the cellulose-processing functionality in a host organism is disclosed. Such method may include the steps of (a) modifying at least one polynucleotide encoding a protein, wherein the at least one polynucleotide has at least two repeat sequences within the coding region, and said at least two repeat sequences have 100% nucleotide sequence identity over a continuous stretch of at least 20 nucleotide in length; and (b) introducing said at least one polynucleotide into said host organism. In one aspect, the modifying step (a) includes mutating the at least one polynucleotide to eliminate the at least two repeat sequences on the polynucleotide without altering the sequence of the protein encoded by the at least one polynucleotide; Such a method can be applicable on many different hosts, such as bacteria and fungi, and more preferably, Thermoanaerobacterium saccharolyticum. The mutagenesis step may further include codon optimization such that the mutated coding sequence is more suitable for gene expression in the host organism. In a preferred embodiment, said at least one polynucleotide encodes one or more members of the bacteria cellulosomal proteins.

In another embodiment, a method for producing ethanol includes generating an organism containing at least one modified scaffoldin gene and at least one cellulase gene, incubating the organism in a medium containing at least one substrate selected from the group consisting of glucose, xylose, mannose, arabinose, galactose, fructose, cellobiose, sucrose, maltose, xylan, mannan, starch, cellulose, pectin and combinations thereof to allow for production of ethanol from the substrate.

### BRIEF DESCRIPTION OF THE DRAWINGS

FIG. 1 shows the gene structure of the wild-type cipA from Clostridium thermocellum and the locations of the 10 longest Repeats #1-10 indicated by arrows, with the longest repeat, Repeat #1, shown at the very bottom, and the shortest, Repeat #10, shown at the very top.

FIG. 2 shows sequence alignment between the wild-type cipA gene (wcipA) from Clostridium thermocellum and a modified cipA gene (mcipA) disclosed herein.

FIG. 3 shows the improved genetic stability of the modified cipA (mcipA) gene as compared to the wild-type cipA gene.

### DETAILED DESCRIPTION

There will now be shown and described improved methods for creating and utilizing thermophilic bacteria in the conversion of biomass to ethanol.

As used herein, an organism is in “a native state” if it has not been genetically engineered or otherwise manipulated by the hand of man in a manner that alters the genotype and/or phenotype of the organism. A gene or a protein is considered to be “wild-type” if its sequence is identical to the ones isolated from an organism in its native state.

“Identity” refers to a comparison between sequences of polynucleotide or polypeptide molecules. Methods for determining sequence identity are commonly known. Computer programs typically employed for performing an identity comparison include, for example, the Gap program (Wisconsin Sequence Analysis Package, Version 8 for Unix, Genetics Computer Group, University Research Park, Madison Wis.), which uses the algorithm of Smith and Waterman (1981) Adv. Appl. Math. 2: 482-489.

For purpose of this disclosure, the term “repeat” or “repeat sequence” refers to a fragment or a stretch of sequence with a minimum length of 10 by which is contained within a larger polynucleotide molecule (DNA or RNA), wherein the sequence of said fragment is at least 99% identical with one or more fragment or stretch of sequence contained within the same molecule. These fragments of sequences can be said to belong to a repeat sequence group. The term “removing a repeat sequence” or “eliminating a repeat sequence” means that one or more of the sequences within a repeat sequence group are modified such that no fragments longer than 10 bp within said repeat sequence group share more than 99% sequence identify with one another. Repeat sequences may be removed by cutting out one or more such fragments, by mutations that decrease the sequence identity among the repeat sequences, or by point mutations that disrupt the continuity of homologous sequences. Ideally, for a molecule such as the cipA gene which possesses multiple repeat sequence groups, all repeat fragments in each repeat sequence group shall be removed (or eliminated). Practically, however, this task may be hard to accomplish without causing changes to the sequence of the encoded protein. As shown in the Examples below and in SEQ ID NO: 2, most of the repeat groups have been removed, but some repeat groups remain. In some cases, new repeat groups may be generated during the process of removing other repeat groups.

“Lignocellulosic substrate” generally refers to any lignocellulosic biomass suitable for use as a substrate to be converted into ethanol.

“Saccharification” refers to the process of breaking a complex carbohydrate, such as starch or cellulose, into its monosaccharide or oligosaccharide components. For purposes of this disclosure, a complex carbohydrate is preferably processed into its monosaccharide components during a saccharification process.

The term “endogenous” is used to describe a molecule that exists naturally in an organism. A molecule that is introduced into an organism using molecular biology tools, such as transgenic techniques, is not endogenous to that organism.

Techniques for mutation of a gene may include, but are not limited to, deletion, insertion, substitution in the coding or non-coding regulatory sequences of the target gene, as well as the use of RNA interference to suppress gene expression.

For purposes of this disclosure, an organism that possesses the necessary biological and chemical components, including polynucleotides, polypeptides, carbohydrates, lipids and other molecules, as well as cellular or subcellular structures that may be required for performing or facilitating certain biological and/or chemical processes is deemed to be capable of performing said processes. Thus, an organism that contains certain inducible genes may be considered capable of performing the function attributable to the protein encoded by those genes.

The term “genetic engineering” is used to refer to a process by which genetic materials, including DNA and/or RNA, are manipulated in a cell or introduced into a cell to affect expression of certain proteins in said cell. Manipulation may include introduction of a foreign (or “exogenous”) gene into the cell or inactivation or modification of an endogenous gene. Such a modified cell may be called a “genetically engineered cell” or a “genetically modified cell.” If the original cell to be genetically engineered is a bacterial cell, said genetically engineered cell may be said to have been derived from a bacterial cell. A molecule that is introduced into a cell to genetically modify the cell may be called a genetic construct. A genetic construct typically carries one or more DNA or RNA sequences on a single molecule.

The expression of a protein is generally regulated by a non-coding region of a gene termed a promoter. When a promoter controls the transcription of a gene, it can also be said that the expression of the gene (or the encoded protein) is driven by the promoter. When a promoter is placed in proximity of a coding sequence, such that transcription of the coding sequence is under control of the promoter, it can be said that the coding sequence is operably linked to the promoter. A promoter that is not normally associated with a gene is called a “heterologous promoter.” The expression of a gene in a microorganism which does not normally express such a gene is called “heterologous expression.”

A “cellulolytic material” is a material that may facilitate the breakdown of cellulose into its component oligosaccharides or monosaccharides. For example, cellulolytic material may comprise a cellulase or hemicellulase.

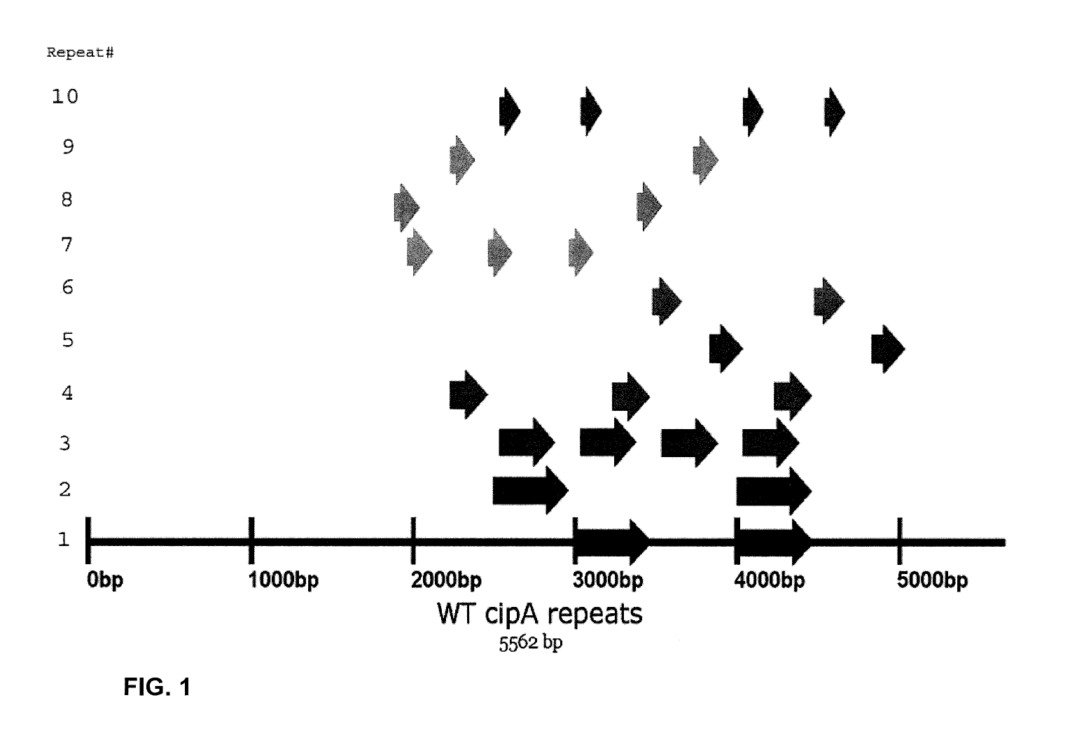

The coding sequence of the cipA gene of C. thermocellum contains extensive areas of repeated sequences (FIG. 1), which may render the gene unstable. The reason for this instability is the propensity of these large repeated regions to cause errors in PCR as well as the creation of truncated cipA due to homologous recombination in yeast, E. coli (both used for cloning purposes), or T. saccharolyticum.

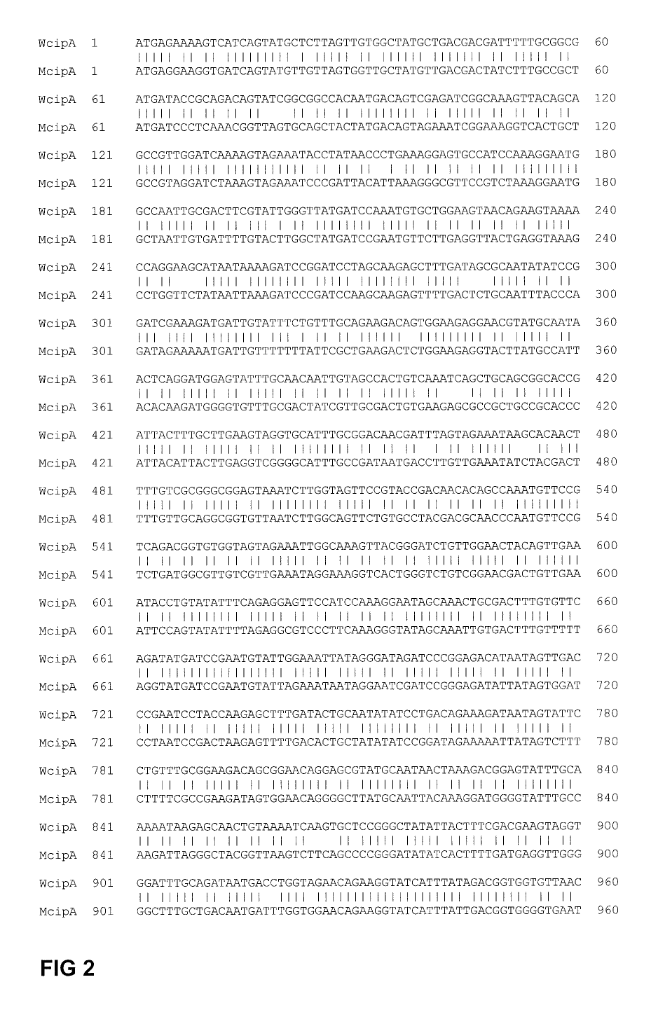

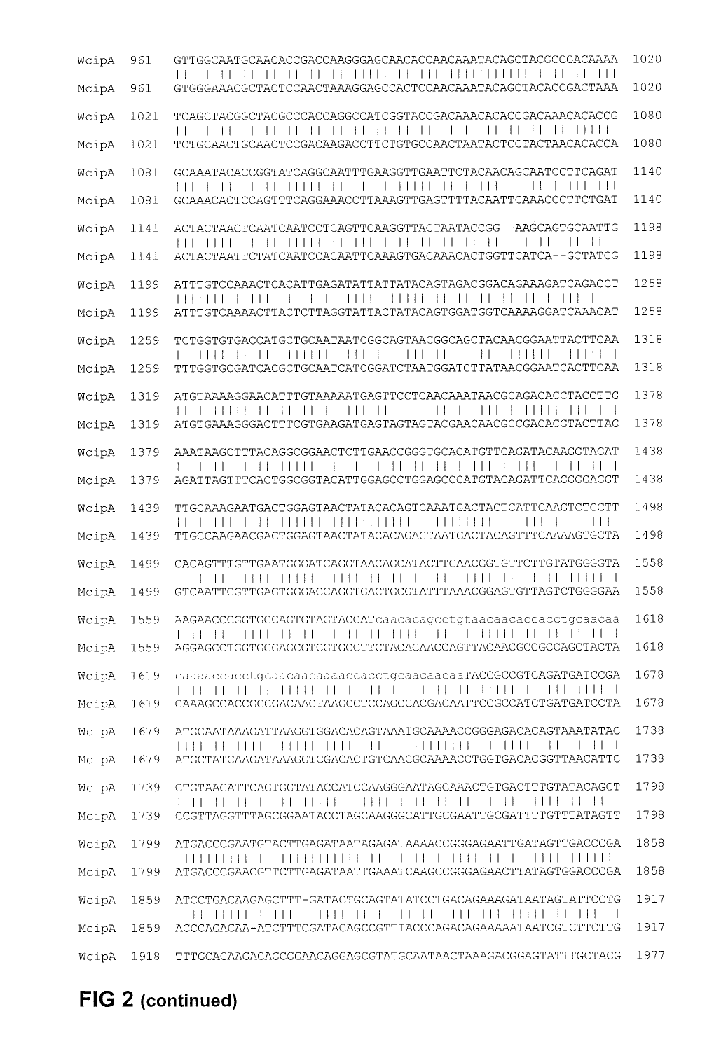

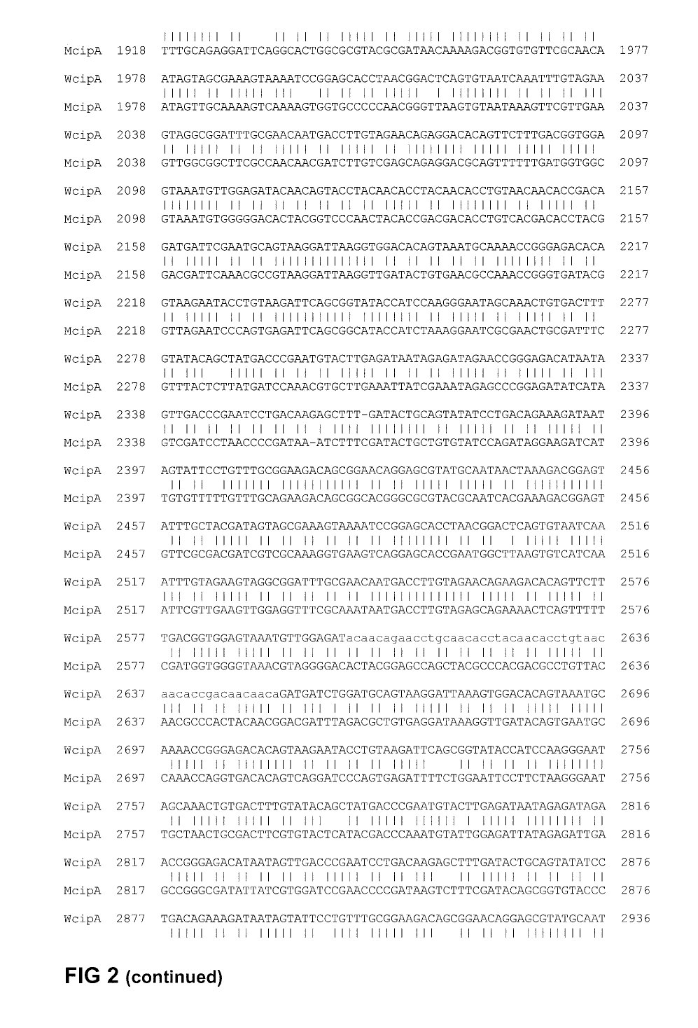

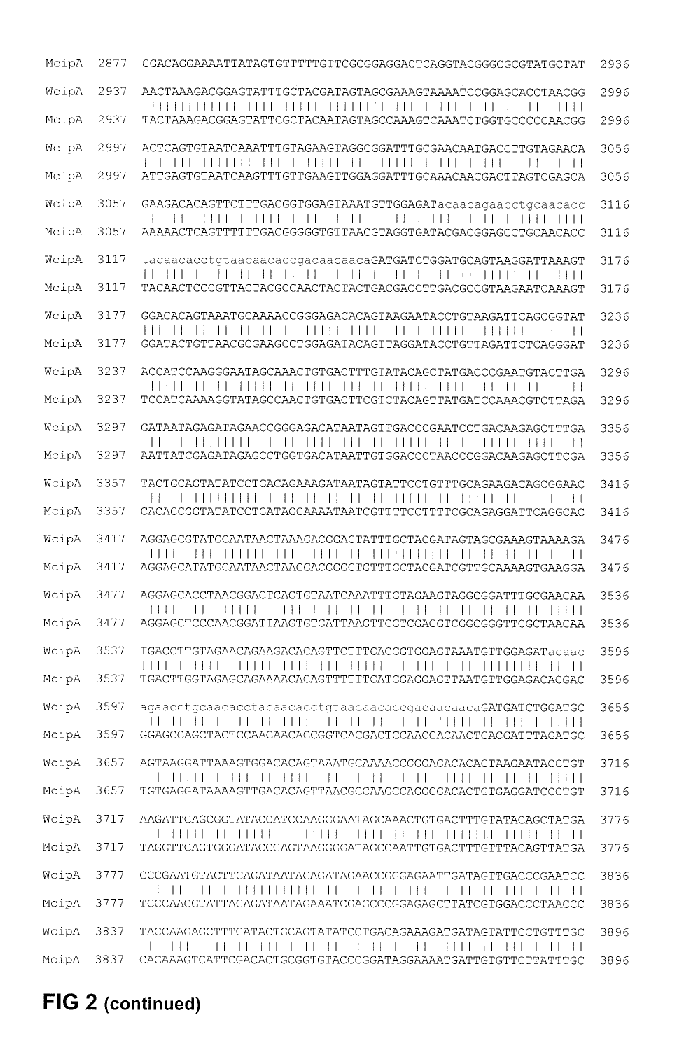

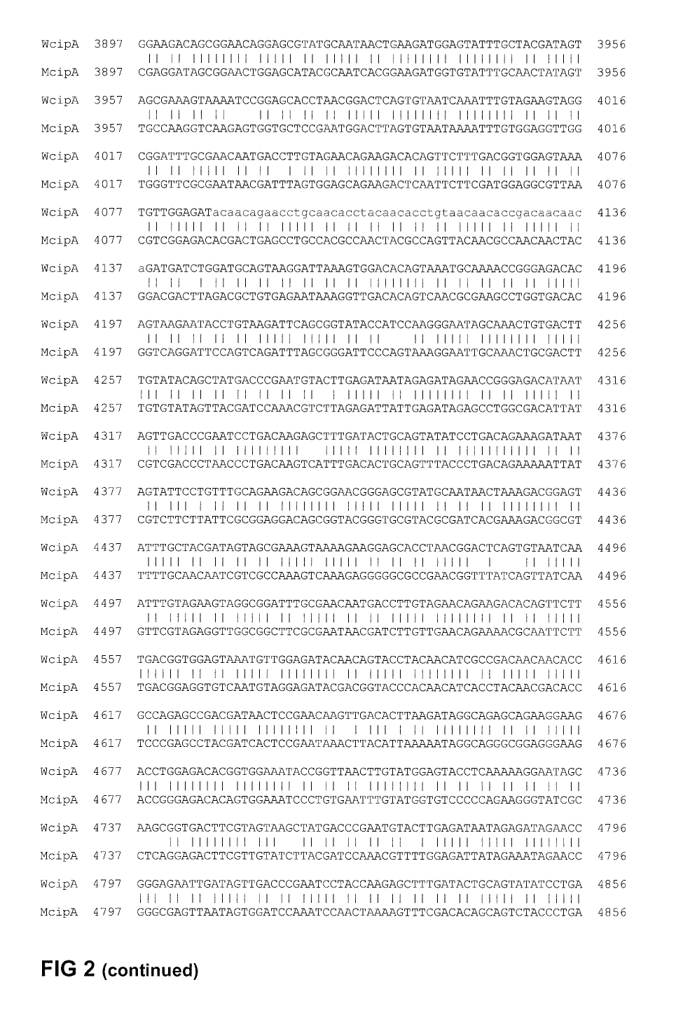

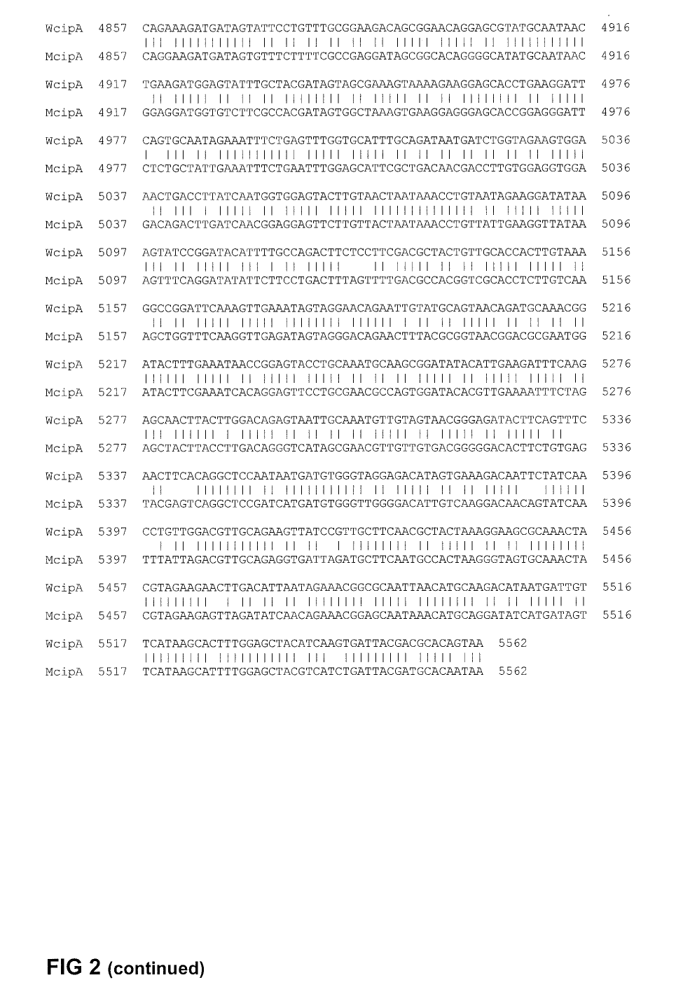

The ten longest repeats on the cipA gene of C. thermocellum are listed in Table 1 and are shown schematically in FIG. 1. As disclosed herein, these repeated sequences may be eliminated to create a modified cipA gene that is more stable when transformed into various organisms. It is one objective of the present disclosure to create a modified cipA gene (“mcipA”) encoding a CipA protein that is identical, or substantially similar in amino acid sequence to the wildtype CipA protein of C. thermocellum. One such mcipA gene (SEQ ID NO: 2) is shown in FIG. 2 in a sequence alignment with the wildtype cipA gene (SEQ ID NO: 1). As shown in FIG. 2, these two sequences exhibit about 76% sequence identity. It is to be understood that even the mcipA sequence (SEQ ID NO: 2) contains some repeat sequences, but the sequence similarity among the repeat sequences in mcipA is significantly lower than that of the unmodified cipA gene from C. thermocellum. It is also worth noting that the mcipA gene may be modified to further reduce the length and sequence similarity of the repeat sequence.

In one embodiment of the present disclosure, alternative codons that are more commonly used in the intended host strain may be utilized to modify the cipA gene. Codon usage generally reflects the availability of tRNA isoforms in different organisms. Efficiently expressed genes typically utilize the most abundant tRNA isoforms in the organism. For instance, certain codons are used unproportionally more frequently than others in some strains of Thermoanaerobacterium saccharolyticum. See e.g., Lee Y E, Ramesh M V, Zeikus J G, Cloning, sequencing and biochemical characterization of xylose isomerase from Thermoanaerobacterium saccharolyticum strain B6A-R1. J Gen Microbiol. 1993 June; 139 Pt 6:1227-34. By designing a modified cipA gene utilizing alternative codons that are used in T. saccharolyticum at least 10% of the time, it is possible to create a cipA gene which is codon-optimized for T. saccharolyticum and which also has its longest repeated region shortened to less than 19 bp. The net result is a cipA gene that is well suited for expression in an organism that shares similar codon biases as T. saccharolyticum. The mcipA gene thus generated tends to be substantially more stable than the wildtype counterpart, which opens up the possibility of producing full sized cellulosomes in T. saccharolyticum as well as in other organisms where stability of cipA may be problematic.

It is to be recognized that the codon optimization can be tailored to any organism desired as an intended host for expression of the cipA gene. Briefly, the sequence of the gene from a first organism is determined. If this gene is to be expressed heterologously in a second organism, the codon usage of the second organism is then determined by comparing the codon usage frequency of that second organism with the codon usage frequency of the first organism. The sequence of the gene can then be modified such that the usage of the codon is biased towards the second organism. It may also be desirable to apply the codon modification/optimization disclosed herein to genes encoding other structural and/or catalytic subunits of the cellulosomes or other cellular proteins. Codon modification may be performed by DNA or RNA synthesis, PCR, cloning, other molecular cloning techniques, and combination thereof. See generally, J. Sambrook, Molecular Cloning: A Laboratory Manual (3-Volume Set), Cold Spring Harbor Laboratory Press (Jan. 15, 2001).

The modified cipA gene may be introduced into a host organism. Examples of such host organisms may include but are not limited to thermophilic bacteria capable of digesting cellulose. More preferably, the host bacteria are gram positive bacteria other than C. thermocellum. Most preferably, the host bacterium is Thermoanaerobacterium saccharolyticum.

The thermophilic bacterium, T. saccharolyticum, is used by way of example to illustrate how cipA and cellulosome activities in an organism may be manipulated to enhance biomass digestion and ethanol production. The methods and materials disclosed herein may however apply to members of the Thermoanaerobacter and Thermoanaerobacterium genera, as well as other microorganisms. Members of the Thermoanaerobacter and Thermoanaerobacterium genera may include, for example, Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and/or progeny thereof. The cipA and cellulosome modification approaches for maximizing ethanol production from biomass may be applicable in genetic engineering of other microorganisms, such as yeast or fungi.

Major groups of bacteria include eubacteria and archaebacteria. Thermophilic eubacteria include: phototropic bacteria, such as cyanobacteria, purple bacteria and green bacteria; Gram-positive bacteria, such as Bacillus, Clostridium, lactic acid bacteria and Actinomyces; and other eubacteria, such as Thiobacillus, Spirochete, Desulfotomaculum, Gram-negative aerobes, Gram-negative anaerobes and Thermotoga. Within archaebacteria are considered Methanogens, extreme thermophiles (an art-recognized term) and Thermoplasma. In certain embodiments, the present instrumentalities relate to Gram-negative organotrophic thermophiles of the genus Thermus; Gram-positive eubacteria, such as Clostridium, which comprise both rods and cocci; eubacteria, such as Thermosipho and Thermotoga; archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus and Methanopyrus. Some examples of thermophilic or mesophilic organisms (including bacteria, prokaryotic microorganisms and fungi), which may be suitable for use with the disclosed instrumentalities include, but are not limited to: Anaerocellum sp., Thermoanaerobacterium thermosaccharolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus Havas, Thermus ruber, Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinata, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophila, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogena, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, variants thereof, and/or progeny thereof.

In one aspect, an isolated polynucleotide may comprise the nucleotide sequence of the mcipA gene (SEQ ID NO: 2) or fragment thereof. Alternatively, a polynucleotide may have substantial sequence similarity to SEQ ID NO: 2, for example, with at least 80%, 90%, 95%, 98%, or 99% sequence identity to the sequence of SEQ ID NO: 2. In another aspect, a polynucleotide may have substantial sequence similarity to SEQ ID NO: 1, for example, with at least 70%, 80%, 90%, 95%, 98%, or 99% sequence identity to the sequence of SEQ ID NO: 1, said polynucleotide may have at least one of the repeats selected from the group consisting of Repeat #1-10 removed. It is to be understood that the same repeat group may be eliminated using different methods, which may result in different modified versions of the cipA gene. These variants of mcipA gene are within the scope of the present invention.

In another aspect, a polynucleotide may encode a protein or a fragment thereof with substantially the same or similar activity as the scaffoldin protein or fragment thereof encoded by SEQ ID NO: 1, wherein the polynucleotide sequence may have at least 70%, 80%, 90%, 95%, 98%, or 99% sequence identity with the corresponding sequence of SEQ ID NO: 1, said polynucleotide may have at least one of the repeats selected from the group consisting of Repeat #1-10 removed. In yet another aspect, a vector comprising a polynucleotide of SEQ ID NO: 2 is disclosed.

For purpose of this disclosure, the cipA scaffoldin protein, encoded by the cipA gene, may be referred to as “cipA protein,” “cipA subunit,” or “CipAp” (SEQ ID NO: 3). It is conceivable that a protein with substantial sequence similarity to SEQ ID NO: 3 may have substantially similar functionality or activity as the corresponding cipA subunit. For purpose of this disclosure, other proteins capable of serving as a scaffolding protein for the assembly of cellulosomes and sharing at least about 70% sequence identity with the protein of SEQ ID NO: 3 may be used to function in place of the cipA protein of SEQ ID NO: 3. More preferably, such proteins share at least 80%, 90%, 95%, 98% or 99% sequence identity with SEQ ID NO: 3 and possess substantially the same or similar functionality as the cipA protein of SEQ ID NO: 3.

The codon shifted and sequence heterogenized cipA gene, when introduced into a host organism such as T. saccharolyticum, may enhance the conversion of biomass to ethanol because the mcipA optimized for T. saccharolyticum expression may be better suited for expression in T. saccharolyticum or any other host that shares the same or similar codon biases as T. saccharolyticum. The mcipA gene may also be more stable than the unmodified wildtype gene in the transformed organism.

It will be appreciated that carbohydrate-rich biomass material that is saccharified to produce one or more of glucose, xylose, mannose, arabinose, galactose, fructose, cellobiose, sucrose, maltose, xylan, mannan, starch cellulose and pectin may be utilized by the disclosed organisms. In various embodiments, the biomass may be lignocellulosic biomass that comprises wood, corn stover, sawdust, bark, leaves, agricultural and forestry residues, grasses such as switchgrass, ruminant digestion products, municipal wastes, paper mill effluent, newspaper, cardboard, or combinations thereof.

### Deposit

Modified T. saccharolyticum containing the mcipA gene will be deposited with the American Type Culture Collection, Manassas, Va. 20110-2209. This deposit will be made in compliance with the Budapest Treaty requirements that the duration of the deposit should be for thirty (30) years from the date of deposit or for five (5) years after the last request for the deposit at the depository or for the enforceable life of a U.S. Patent that matures from this application, whichever is longer. Modified T. saccharolyticum will be replenished should it become non-viable at the depository.

The following examples illustrate the present invention. These examples are provided for purposes of illustration only and are not intended to be limiting. The chemicals and other ingredients are presented as typical components or reactants, and various modifications may be derived in view of the foregoing disclosure within the scope of the invention.

### Example 1

### Codon Shifting and Sequence Heterogenation of the cipA Gene from Clostridium thermocellum

Clostridium thermocellum is a thermophilic, anaerobic bacterium. The cipA gene of C. thermocellum may be isolated by standard cloning techniques. More specifically, the cipA gene can be amplified by PCR using primers that contain cloning sites. The amplified product may then be subcloned into a vector using standard recombinant DNA technology. PCR and/or restriction digestion by enzymes may be utilized to remove the repeated sequences.

In one aspect, synthetic oligonucleotides carrying one or more point mutations may be prepared and used as primers to amplify certain segments of the cipA gene. These amplified segments may then be annealed together to form a modified cipA gene lacking the major repeat sequences that are present in the wild-type cipA gene (SEQ ID No. 1).

In one preferred embodiment, the entire coding sequence of the wild-type cipA gene from Clostridium thermocellum was examined to identify major repeat sequences. A modified version of the cipA gene (mcipA) was designed to remove major repeat sequences and to optimize for codon usage in Thermoanaerobacterium saccharolyticum without altering the sequence of the encoded protein. The whole-gene synthesis of mcipA was performed by GeneArt, Inc. (Burlingame, Calif. 94010). The sequence of mcipA (SEQ ID No. 2) lacked major repeat sequences that are present in the wild-type cipA gene (SEQ ID No. 1). The longest repeat in the mcipA gene was a 19-bp repeat sequence, which was much shorter than the length of the repeated sequences in the unmodified wild-type gene. As a result, the mcipA was genetically more stable when transformed into various organisms, such as T. saccharolyticum.

Alternative codons that are more commonly used in the intended host strain may also be utilized to modify the cipA gene. For instance, it is known that certain genes from certain strains of Thermoanaerobacterium saccharolyticum show codon biases in that certain codons are unproportionally used more frequently than others. See e.g., Lee Y E, Ramesh M V, Zeikus J G, Cloning, sequencing and biochemical characterization of xylose isomerase from Thermoanaerobacterium saccharolyticum strain B6A-RI. J Gen Microbiol. 1993 June; 139 Pt 6:1227-34.

By designing a modified cipA gene utilizing alternative codons that are used in T. saccharolyticum at least 10% of the time, it is possible to create a cipA which is codon optimized for T. saccharolyticum and which has relatively fewer and shorter repeated regions. The synthesized mcipA (SEQ ID No. 2) had codon usage that was optimized by expression in Thermoanaerobacterium saccharolyticum and would have higher efficiency in gene expression when transformed into T. saccharolyticum. The net result is a modified cipA gene that was well suited for expression in any organism with similar codon biases as the intended host, such as T. saccharolyticum.

### Example 2

### Introduction of the Modified cipA Gene into Thermoanaerobacterium saccharolyticum

Thermoanaerobacterium saccharolyticum

Thermoanaerobacterium saccharolyticum is a thermophilic, anaerobic bacterial species. The strain JW/SL-YS485 (DSM 8691) was isolated from the West Thumb Basin in Yellowstone National Park, Wyoming. (Lui, S. Y., F. C. Gherardini, M. Matuschek, H. Bahl, J. Wiegel (1996) Cloning, sequencing, and expression of the gene encoding a large S-layer-associated endoxylanase from Thermoanaerobacterium sp strain JW/SL-YS485 in Escherichia coli. J. Bacteriol. 178: 1539-1547; Mai, V., J. Wiegel (2000) Advances in development of a genetic system for Thermoanaerobacterium spp: Expression of genes encoding hydrolytic enzymes, development of a second shuttle vector, and integration of genes into the chromosome. Appl. Environ. Microbiol. 66: 4817-4821, 2000.) It grows at a temperature range of 30-66° C. and a pH range of 3.85-6.5. It consumes a variety of biomass derived substrates including the monosaccharides glucose and xylose, the disaccharides cellobiose and sucrose, and the polysaccharides xylan and starch. The organism produces ethanol as well as the organic acids lactic acid and acetic acid as primary fermentation products.

Transformation of T. saccharolyticum

Transformation of T. saccharolyticum can be performed at least with the following two methods. The first method is as previously described by Mai, V., Lorenz, W. W. and J. Wiegel. (1997) Transformation of Thermoanaerobacterium sp. strain JW/SL-YS485 with plasmid pIKM1 conferring kanamycin resistance. FEMS Microbiol. Lett. 148: 163-167.). The second method has several modifications following cell harvest and is based on the method developed for Clostridium thermocellum. (Tyurin, M. V., S. G. Desai, L. R. Lynd, (2004) Electrotransformation of Clostridium thermocellum. Appl. Environ. Microbiol. 70(2): 883-890.)

Briefly, cells are grown overnight using pre-reduced medium DSMZ 122 in sterile disposable culture tubes inside an anaerobic chamber in an incubator maintained at 55° C. Thereafter, cells are sub-cultured with 4 μg/ml isonicotonic acid hydrazide (isoniacin), a cell wall weakening agent (Hermans, J., J. G. Boschloo, J. A. M. de Bont (1990), FEMS Microbiol. Lett. 72: 221-224) added to the medium after the initial lag phase. Exponential phase cells are harvested and washed with pre-reduced cold sterile 200 mM cellobiose solution, and resuspended in the same solution and kept on ice. Cells are kept cold (approximately 4° C.) during this process.

Samples composed of 90 μl of the cell suspension and 2 to 6 μl of the knockout or control vector (1 to 3 μg) added just before pulse application, are placed into sterile 2 ml polypropylene microcentrifuge disposable tubes that served as electrotransformation cuvettes. A square-wave with pulse length set at 10 ms is applied using a custom-built pulse generator/titanium electrode system. A voltage threshold corresponding to the formation of electropores in a cell sample is evaluated as a non-linear current change when pulse voltage is linearly increased in 200V increments. A particular voltage that provided the best ratio of transformation yield versus cell viability rate at a given DNA concentration is used. The voltage used in this experiment can be set at 25 kV/cm. Pulsed cells are initially diluted with 500 μl DSM 122 medium, held on ice for 10 minutes and then recovered at 55° C. for 4-6 hrs. Following recovery, cells transformed with the control vector are mixed with medium containing 1% agar and either kanamycin at 200 μg/ml or erythromycin at 10 μg/ml and poured onto petri plates with media at pH 6.7 for kanamycin selection or pH 6.1 for erythromycin selection and incubated in anaerobic jars for 4 days at 52° C. Other media that can support growth of T. saccharolyticum may also be used. The transformed cell lines may be used without further manipulation. Subsequent transformation may be carried out as described above with the primary transformant substituted for the non-transformed cell suspension.

T. saccharolyticum strains with the mcipA gene may be created by transformation of wild-type T. saccharolyticum with appropriate constructs as described above. The modified cipA gene may be carried on a vector that is capable of self-replicating and can exist independently of the chromosomes of the host. Alternatively, the modified cipA gene may be carried on a vector that facilitates integration of the mcipA gene onto the chromosomes of the host. The mcipA gene thus generated is substantially more stable than the wildtype cipA gene in T. saccharolyticum. The expression of the modified CipA protein may also be more efficient in T. saccharolyticum than that of the wildtype CipA protein from Clostridium thermocellum.

### Example 3

### Improved Genetic Stability of the mcipA Gene in Thermoanaerobacterium saccharolyticum as Compared to Wild-Type cipA Gene

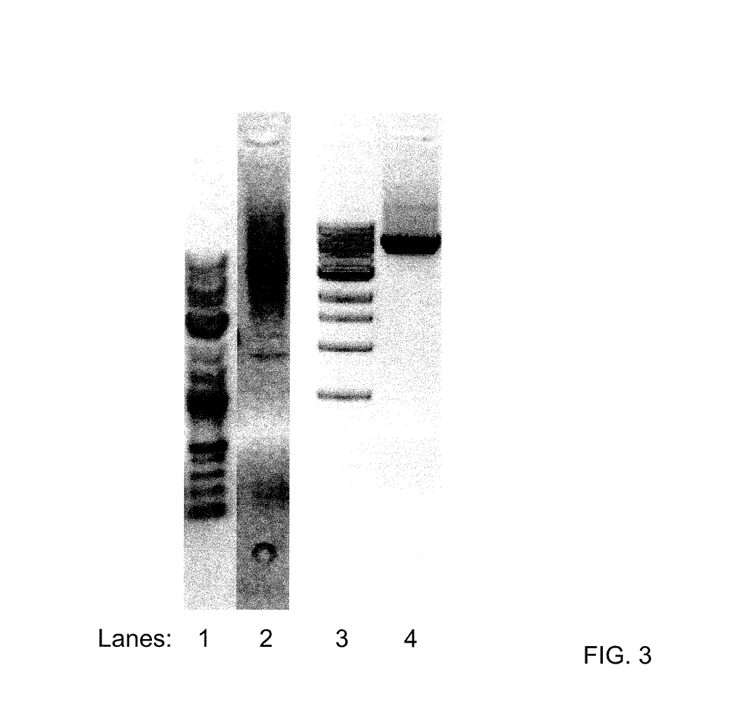

The modified cipA gene (mcipA) synthesized by GeneArt as described in Example 1 was introduced into Thermoanaerobacterium saccharolyticum according to transformation methods described in Example 2. The plasmid carrying the mcipA gene was stable in the T. saccharolyticum host. Total genomic DNA was prepared from the transformed T. saccharolyticum strain and used as template to amplify the mcipA in a PCR reaction using primers that amplify the full-length cipA gene. Total genomic DNA was also prepared from a Clostridium thermocellum strain and used as template to amplify the wild-type cipA in a PCR. The PCR products were analyzed on agarose gels along with size markers. As shown in FIG. 3, while the PCR product of the modified cipA gene showed a clear and distinct band (lane 4), the PCR product of the wild-type cipA gene from C. thermocellum showed a long smear (lane 2). Lanes 1 and 3 are both 1 kilobase ladder (New England Biolabs). These results suggest that the modified cipA gene has significantly improved genetic stability and is much easier to clone and manipulate in host organisms such as yeast, E. coli, and T. saccharolyticum, as compared to the wild-type cipA gene from C. thermocellum.

### Example 4

### Introduction of Other Genes into Thermoanaerobacterium saccharolyticum

Other cellulosome components can be introduced into Thermoanaerobacterium saccharolyticum to build the cellulosomes. Structural or non-catalytic components, such as the anchor proteins, may be introduced. Enzymatic component of the cellulosomes, such as those known to be present in the cellulosome of Clostridium thermocellum, may be introduced into the host strain. One Example of such anchor protein may be the sdbA gene. Enzymes may be introduced into the host strain to build the cellulosome. Alternatively, the enzyme may be introduced into T. saccharolyticum once at a time, so that the synergistic effects of these various enzyme can be evaluated. The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or terminology employed herein is for the purpose of description and not limitation. Certain terms with capital or small letters, in singular or in plural forms, may be used interchangeably in this disclosure.

All references mentioned in this application are incorporated by reference to the same extent as though fully replicated herein.

What is claimed is:

1. An isolated polynucleotide having at least 70% sequence identity to the polynucleotide of SEQ ID. NO. 1, wherein at least one Repeat Group selected from Repeat Groups 1-10 in the polynucleotide of SEQ ID. NO. 1 has been eliminated.

2. The polynucleotide of claim 1 having at least 80% sequence identity to the polynucleotide of SEQ ID. NO. 1.

3. The polynucleotide of claim 1 having at least 90% sequence identity to the polynucleotide of SEQ ID. NO. 1.

4. The polynucleotide of claim 1 having at least 99% sequence identity to the polynucleotide of SEQ ID. NO. 1.

5. An isolated polynucleotide having at least 80% sequence identity to the polynucleotide of SEQ ID. NO. 2.

6. The polynucleotide of claim 1 having at least 90% sequence identity to the polynucleotide of SEQ ID. NO. 2.

7. The polynucleotide of claim 1 having at least 95% sequence identity to the polynucleotide of SEQ ID. NO. 2.

8. An isolated polynucleotide having the sequence of SEQ ID. NO. 2.

9. A genetic construct comprising the polynucleotide of claim 1, said polynucleotide being operably linked to a promoter, wherein said promoter is capable of regulating gene expression from said polynucleotide.

10. The genetic construct of claim 9, wherein said promoter enhances gene expression from said polynucleotide in Thermoanaerobacterium saccharolyticum.

11. An organism capable of growing on a carbohydrate-rich biomass substrate, said organism comprising the polynucleotide of claim 1, 5 or 8, wherein said organism is capable of expressing a scaffolding protein encoded by said polynucleotide.

12. The organism of claim 11, wherein said organism is Thermoanaerobacterium saccharolyticum.

13. A method for improving the cellulose-processing functionality in a host organism, said method comprising the steps of:
(a) modifying at least one polynucleotide encoding a protein, said at least one polynucleotide having at least two repeat sequences within the coding region, said at least two repeat sequences having 100% nucleotide sequence identity over a continuous stretch of at least 20 nucleotide in length, wherein the modifying step comprises mutating said at least one polynucleotide to eliminate said at least two repeat sequences on said at least one polynucleotide without changing the sequence of the protein encoded by said at least one polynucleotide; and
(b) introducing said at least one polynucleotide into said host organism.

14. The method of claim 13, wherein said organism is Thermoanaerobacterium saccharolyticum.

15. The method of claim 13, wherein said at least one polynucleotide is mutated so that the codon usage is optimized for the host organism.

16. The method of claim 13, further comprising the step of expressing said polypeptide encoded by said at least one polynucleotide in said organism.

17. The method of claim 13, wherein the at least one polynucleotide encodes at least one member of the bacterial cellulosome.

18. An organism generated according to the method of claim 13.


SEQUENCE LISTING (3 sequences)

SEQ ID NO: 1 - DNA, 5562 bp, Clostridium thermocellum
atgagaaaagtcatcagtatgctcttagttgtggctatgctgacgacgatttttgcggcgatgataccgcagacagtatcggcggccacaatgacagtcgagatcggcaaagttacagcagccgttggatcaaaagtagaaatacctataaccctgaaaggagtgccatccaaaggaatggccaattgcgacttcgtattgggttatgatccaaatgtgctggaagtaacagaagtaaaaccaggaagcataataaaagatccggatcctagcaagagctttgatagcgcaatatatccggatcgaaagatgattgtatttctgtttgcagaagacagtggaagaggaacgtatgcaataactcaggatggagtatttgcaacaattgtagccactgtcaaatcagctgcagcggcaccgattactttgcttgaagtaggtgcatttgcggacaacgatttagtagaaataagcacaacttttgtcgcgggcggagtaaatcttggtagttccgtaccgacaacacagccaaatgttccgtcagacggtgtggtagtagaaattggcaaagttacgggatctgttggaactacagttgaaatacctgtatatttcagaggagttccatccaaaggaatagcaaactgcgactttgtgttcagatatgatccgaatgtattggaaattatagggatagatcccggagacataatagttgacccgaatcctaccaagagctttgatactgcaatatatcctgacagaaagataatagtattcctgtttgcggaagacagcggaacaggagcgtatgcaataactaaagacggagtatttgcaaaaataagagcaactgtaaaatcaagtgctccgggctatattactttcgacgaagtaggtggatttgcagataatgacctggtagaacagaaggtatcatttatagacggtggtgttaacgttggcaatgcaacaccgaccaagggagcaacaccaacaaatacagctacgccgacaaaatcagctacggctacgcccaccaggccatcggtaccgacaaacacaccgacaaacacaccggcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagtaccatcaacacagcctgtaacaacaccacctgcaacaacaaaaccacctgcaacaacaaaaccacctgcaacaacaataccgccgtcagatgatccgaatgcaataaagattaaggtggacacagtaaatgcaaaaccgggagacacagtaaatatacctgtaagattcagtggtataccatccaagggaatagcaaactgtgactttgtatacagctatgacccgaatgtacttgagataatagagataaaaccgggagaattgatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaggacacagttctttgacggtggagtaaatgttggagatacaacagtacctacaacacctacaacacctgtaacaacaccgacagatgattcgaatgcagtaaggattaaggtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcggaagacagcggaacaggagcgtatgcaataactaaagacggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcagaagacagcggaacaggagcgtatgcaataactaaagacggagtatttgctacgatagtagcgaaagtaaaagaaggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagaattgatagttgacccgaatcctaccaagagctttgatactgcagtatatcctgacagaaagatgatagtattcctgtttgcggaagacagcggaacaggagcgtatgcaataactgaagatggagtatttgctacgatagtagcgaaagtaaaatccggagcacctaacggactcagtgtaatcaaatttgtagaagtaggcggatttgcgaacaatgaccttgtagaacagaagacacagttctttgacggtggagtaaatgttggagatacaacagaacctgcaacacctacaacacctgtaacaacaccgacaacaacagatgatctggatgcagtaaggattaaagtggacacagtaaatgcaaaaccgggagacacagtaagaatacctgtaagattcagcggtataccatccaagggaatagcaaactgtgactttgtatacagctatgacccgaatgtacttgagataatagagatagaaccgggagacataatagttgacccgaatcctgacaagagctttgatactgcagtatatcctgacagaaagataatagtattcctgtttgcagaagacagcggaacgggagcgtatgcaataactaaagacggagtatttgctacgatagtagcgaaagtaaaagaaggagcacctaacggactcagtgtaatcaaatttgtagaagtaggcggatttgcgaacaatgaccttgtagaacagaagacacagttctttgacggtggagtaaatgttggagatacaacagtacctacaacatcgccgacaacaacaccgccagagccgacgataactccgaacaagttgacacttaagataggcagagcagaaggaagacctggagacacggtggaaataccggttaacttgtatggagtacctcaaaaaggaatagcaagcggtgacttcgtagtaagctatgacccgaatgtacttgagataatagagatagaaccgggagaattgatagttgacccgaatcctaccaagagctttgatactgcagtatatcctgacagaaagatgatagtattcctgtttgcggaagacagcggaacaggagcgtatgcaataactgaagatggagtatttgctacgatagtagcgaaagtaaaagaaggagcacctgaaggattcagtgcaatagaaatttctgagtttggtgcatttgcagataatgatctggtagaagtggaaactgaccttatcaatggtggagtacttgtaactaataaacctgtaatagaaggatataaagtatccggatacattttgccagacttctccttcgacgctactgttgcaccacttgtaaaggccggattcaaagttgaaatagtaggaacagaattgtatgcagtaacagatgcaaacggatactttgaaataaccggagtacctgcaaatgcaagcggatatacattgaagatttcaagagcaacttacttggacagagtaattgcaaatgttgtagtaacgggagatacttcagtttcaacttcacaggctccaataatgatgtgggtaggagacatagtgaaagacaattctatcaacctgttggacgttgcagaagttatccgttgcttcaacgctactaaaggaagcgcaaactacgtagaagaacttgacattaatagaaacggcgcaattaacatgcaagacataatgattgttcataagcactttggagctacatcaagtgattacgacgcacagtaa

SEQ ID NO: 2 - DNA, 5562 bp, Artificial
  synthetic DNA
atgaggaaggtgatcagtatgttgttagtggttgctatgttgacgactatctttgccgctatgatccctcaaacggttagtgcagctactatgacagtagaaatcggaaaggtcactgctgccgtaggatctaaagtagaaatcccgattacattaaagggcgttccgtctaaaggaatggctaattgtgattttgtacttggctatgatccgaatgttcttgaggttactgaggtaaagcctggttctataattaaagatcccgatccaagcaagagttttgactctgcaatttacccagatagaaaaatgattgtttttttattcgctgaagactctggaagaggtacttatgccattacacaagatggggtgtttgcgactatcgttgcgactgtgaagagcgccgctgccgcacccattacattacttgaggtcggggcatttgccgataatgaccttgttgaaatatctacgacttttgttgcaggcggtgttaatcttggcagttctgtgcctacgacgcaacccaatgttccgtctgatggcgttgtcgttgaaataggaaaggtcactgggtctgtcggaacgactgttgaaattccagtatattttagaggcgtcccttcaaagggtatagcaaattgtgactttgtttttaggtatgatccgaatgtattagaaataataggaatcgatccgggagatattatagtggatcctaatccgactaagagttttgacactgctatatatccggatagaaaaattatagtctttcttttcgccgaagatagtggaacaggggcttatgcaattacaaaggatggggtatttgccaagattagggctacggttaagtcttcagccccgggatatatcacttttgatgaggttgggggctttgctgacaatgatttggtggaacagaaggtatcatttattgacggtggggtgaatgtgggaaacgctactccaactaaaggagccactccaacaaatacagctacaccgactaaatctgcaactgcaactccgacaagaccttctgtgccaactaatactcctactaacacaccagcaaacactccagtttcaggaaaccttaaagttgagttttacaattcaaacccttctgatactactaattctatcaatccacaattcaaagtgacaaacactggttcatcagctatcgatttgtcaaaacttactcttaggtattactatacagtggatggtcaaaaggatcaaacattttggtgcgatcacgctgcaatcatcggatctaatggatcttataacggaatcacttcaaatgtgaaagggactttcgtgaagatgagtagtagtacgaacaacgccgacacgtacttagagattagtttcactggcggtacattggagcctggagcccatgtacagattcaggggaggtttgccaagaacgactggagtaactatacacagagtaatgactacagtttcaaaagtgctagtcaattcgttgagtgggaccaggtgactgcgtatttaaacggagtgttagtctggggaaaggagcctggtgggagcgtcgtgccttctacacaaccagttacaacgccgccagctactacaaagccaccggcgacaactaagcctccagccacgacaattccgccatctgatgatcctaatgctatcaagataaaggtcgacactgtcaacgcaaaacctggtgacacggttaacattcccgttaggtttagcggaatacctagcaagggcattgcgaattgcgattttgtttatagttatgacccgaacgttcttgagataattgaaatcaagccgggagaacttatagtggacccgaacccagacaaatctttcgatacagccgtttacccagacagaaaaataatcgtcttcttgtttgcagaggattcaggcactggcgcgtacgcgataacaaaagacggtgtgttcgcaacaatagttgcaaaagtcaaaagtggtgcccccaacgggttaagtgtaataaagttcgttgaagttggcggcttcgccaacaacgatcttgtcgagcagaggacgcagttttttgatggtggcgtaaatgtgggggacactacggtcccaactacaccgacgacacctgtcacgacacctacggacgattcaaacgccgtaaggattaaggttgatactgtgaacgccaaaccgggtgatacggttagaatcccagtgagattcagcggcataccatctaaaggaatcgcgaactgcgatttcgtttactcttatgatccaaacgtgcttgaaattatcgaaatagagcccggagatatcatagtcgatcctaaccccgataaatctttcgatactgctgtgtatccagataggaagatcattgtgtttttgtttgcagaagacagcggcacgggcgcgtacgcaatcacgaaagacggagtgttcgcgacgatcgtcgcaaaggtgaagtcaggagcaccgaatggcttaagtgtcatcaaattcgttgaagttggaggtttcgcaaataatgaccttgtagagcagaaaactcagtttttcgatggtggggtaaacgtaggggacactacggagccagctacgcccacgacgcctgttacaacgcccactacaacggacgatttagacgctgtgaggataaaggttgatacagtgaatgccaaaccaggtgacacagtcaggatcccagtgagattttctggaattccttctaagggaattgctaactgcgacttcgtgtactcatacgacccaaatgtattggagattatagagattgagccgggcgatattatcgtggatccgaaccccgataagtctttcgatacagcggtgtacccggacaggaaaattatagtgtttttgttcgcggaggactcaggtacgggcgcgtatgctattactaaagacggagtattcgctacaatagtagccaaagtcaaatctggtgcccccaacggattgagtgtaatcaagtttgttgaagttggaggatttgcaaacaacgacttagtcgagcaaaaaactcagttttttgacgggggtgttaacgtaggtgatacgacggagcctgcaacacctacaactcccgttactacgccaactactactgacgaccttgacgccgtaagaatcaaagtggatactgttaacgcgaagcctggagatacagttaggatacctgttagattctcagggattccatcaaaaggtatagccaactgtgacttcgtctacagttatgatccaaacgtcttagaaattatcgagatagagcctggtgacataattgtggaccctaacccggacaagagcttcgacacagcggtatatcctgataggaaaataatcgttttccttttcgcagaggattcaggcacaggagcatatgcaataactaaggacggggtgtttgctacgatcgttgcaaaagtgaaggaaggagctcccaacggattaagtgtgattaagttcgtcgaggtcggcgggttcgctaacaatgacttggtagagcagaaaacacagttttttgatggaggagttaatgttggagacacgacggagccagctactccaacaacaccggtcacgactccaacgacaactgacgatttagatgctgtgaggataaaagttgacacagttaacgccaagccaggggacactgtgaggatccctgttaggttcagtgggataccgagtaaggggatagccaattgtgactttgtttacagttatgatcccaacgtattagagataatagaaatcgagcccggagagcttatcgtggaccctaaccccacaaagtcattcgacactgcggtgtacccggataggaaaatgattgtgttcttatttgccgaggatagcggaactggagcatacgcaatcacggaagatggtgtatttgcaactatagttgccaaggtcaagagtggtgctccgaatggacttagtgtaataaaatttgtggaggttggtgggttcgcgaataacgatttagtggagcagaagactcaattcttcgatggaggcgttaacgtcggagacacgactgagcctgccacgccaactacgccagttacaacgccaacaactacggacgacttagacgctgtgagaataaaggttgacacagtcaacgcgaagcctggtgacacggtcaggattccagtcagatttagcgggattcccagtaaaggaattgcaaactgcgactttgtgtatagttacgatccaaacgtcttagagattattgagatagagcctggcgacattatcgtcgaccctaaccctgacaagtcatttgacactgcagtttaccctgacagaaaaattatcgtcttcttattcgcggaggacagcggtacgggtgcgtacgcgatcacgaaagacggcgtttttgcaacaatcgtcgccaaagtcaaagagggggcgccgaacggtttatcagttatcaagttcgtagaggttggcggcttcgcgaataacgatcttgttgaacagaaaacgcaattctttgacggaggtgtcaatgtaggagatacgacggtacccacaacatcacctacaacgacacctcccgagcctacgatcactccgaataaacttacattaaaaataggcagggcggagggaagaccgggagacacagtggaaatccctgtgaatttgtatggtgtcccccagaagggtatcgcctcaggagacttcgttgtatcttacgatccaaacgttttggagattatagaaatagaaccgggcgagttaatagtggatccaaatccaactaaaagtttcgacacagcagtctaccctgacaggaagatgatagtgtttcttttcgccgaggatagcggcacaggggcatatgcaataacggaggatggtgtcttcgccacgatagtggctaaagtgaaggagggagcaccggagggattctctgctattgaaatttctgaatttggagcattcgctgacaacgaccttgtggaggtggagacagacttgatcaacggaggagttcttgttactaataaacctgttattgaaggttataaagtttcaggatatattcttcctgactttagttttgacgccacggtcgcacctcttgtcaaagctggtttcaaggttgagatagtagggacagaactttacgcggtaacggacgcgaatggatacttcgaaatcacaggagttcctgcgaacgccagtggatacacgttgaaaatttctagagctacttaccttgacagggtcatagcgaacgttgttgtgacgggggacacttctgtgagtacgagtcaggctccgatcatgatgtgggttggggacattgtcaaggacaacagtatcaatttattagacgttgcagaggtgattagatgcttcaatgccactaagggtagtgcaaactacgtagaagagttagatatcaacagaaacggagcaataaacatgcaggatatcatgatagttcataagcattttggagctacgtcatctgattacgatgcacaataa

SEQ ID NO: 3 - protein, 1853 aa, Clostridium thermocellum
MRKVISMLLVVAMLTTIFAAMIPQTVSAATMTVEIGKVTAAVGSKVEIPITLKGVPSKGMANCDFVLGYDPNVLEVTEVKPGSIIKDPDPSKSFDSAIYPDRKMIVFLFAEDSGRGTYAITQDGVFATIVATVKSAAAAPITLLEVGAFADNDLVEISTTFVAGGVNLGSSVPTTQPNVPSDGVVVEIGKVTGSVGTTVEIPVYFRGVPSKGIANCDFVFRYDPNVLEIIGIDPGDIIVDPNPTKSFDTAIYPDRKIIVFLFAEDSGTGAYAITKDGVFAKIRATVKSSAPGYITFDEVGGFADNDLVEQKVSFIDGGVNVGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAIKIKVDTVNAKPGDTVNIPVRFSGIPSKGIANCDFVYSYDPNVLEIIEIKPGELIVDPNPDKSFDTAVYPDRKIIVFLFAEDSGTGAYAITKDGVFATIVAKVKSGAPNGLSVIKFVEVGGFANNDLVEQRTQFFDGGVNVGDTTVPTTPTTPVTTPTDDSNAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGELIVDPNPTKSFDTAVYPDRKMIVFLFAEDSGTGAYAITEDGVFATIVAKVKSGAPNGLSVIKFVEVGGFANNDLVEQKTQFFDGGVNVGDTTEPATPTTPVTTPTTTDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTVPTTSPTTTPPEPTITPNKLTLKIGRAEGRPGDTVEIPVNLYGVPQKGIASGDFVVSYDPNVLEIIEIEPGELIVDPNPTKSFDTAVYPDRKMIVFLFAEDSGTGAYAITEDGVFATIVAKVKEGAPEGFSAIEISEFGAFADNDLVEVETDLINGGVLVTNKPVIEGYKVSGYILPDFSFDATVAPLVKAGFKVEIVGTELYAVTDANGYFEITGVPANASGYTLKISRATYLDRVIANVVVTGDTSVSTSQAPIMMWVGDIVKDNSINLLDVAEVIRCFNATKGSANYVEELDINRNGAINMQDIMIVHKHFGATSSDYDAQ